(19) United States
(12) Patent Application Publication
(10) Pub. No.: US 2023/0416693 A1
(43) Pub. Date: Dec. 28, 2023

(54) IMPROVED WASHING METHODS FOR AFFINITY CHROMATOGRAPHY

(71) Applicant: Sartorius BIA Separations d.o.o., Ajdovscina (SI)

(72) Inventor: Peter S. GAGNON, Las Vegas, NV (US)

(21) Appl. No.: 18/038,256

(22) PCT Filed: Nov. 24, 2021
(86) PCT No.: PCT/EP2021/082827

§ 371 Date: May 23, 2023

(30) Foreign Application Priority Data
Nov. 24, 2020 (EP) 20209415.7

(51) Int. Cl. C12N 7/00

(52) U.S. Cl. CPC C12N 7/00; C12N 2750/14151

(57) ABSTRACT

A method for removal of contaminants in a sample of AAV comprising the steps of
loading the sample to an affinity chromatography column,
washing the column after the sample is loaded with a first buffer comprising a compound having at least three positive charges or at least three negative charges
employing a second buffer having a salt concentration corresponding to a concentration of at least 0.25 M NaCl for displacing the compound
   wherein neither the compound nor the salt is present in a concentration sufficient to elute the AAV.

The invention pertains to a method for removal of contaminants in a sample of AAV.

### BACKGROUND

The practice of performing washing steps to eliminate weak-binding contaminants before elution of a desired product from a chromatography column is known. In the field of ion exchange chromatography, the simplest example consists of washing a column after sample application with the buffer it was originally equilibrated with. In other cases, the column is washed with a concentration of greater than the equilibration buffer but less than the concentration required to displace the desired product. Other additives may be included in washes so long as they do not inadvertently elute the desired product. These may include surfactants, organic solvents, antiviral compounds, nonionic chaotropes, and extremes of pH, so long as the desired product remains bound to the affinity chromatography column.

Affinity chromatography is more permissive than ion exchange chromatography because its binding is not so dependent on a single retention mechanism. Affinity chromatography ligands often bind their target by means of combined electrostatic, hydrophobic, and hydrogen bonding interactions, the combination of which binds the desired product more strongly than an ion exchanger. In the field of IgG purification with protein A affinity chromatography, secondary washes have been shown to be effective with concentrations of sodium chloride up to 5 M, as well as with urea, organic solvents, surfactants, chelating agents, and reduced pH in various combinations [1-2].

Chromatin in process solutions represents the remnant of the chromosomal mass from dead product-producing cells. It represents a combination of DNA and DNA-binding proteins such as histones organized into nucleosomal arrays. It is documented to interfere with all types of purification media and methods, including filtration, precipitation, and chromatography, including affinity chromatography [3-5]. It binds to all chromatography media. This reduces binding capacity for the desired product while it also inflates protein contamination of the eluted product by 100-fold and inflates DNA contamination by 10,000-fold. Washing affinity columns with concentrated sodium chloride is known to reduce chromatin but does not eliminated it. Chromatin is also known to be highly associated with endotoxins and viruses, if present [6-9].

Adeno-associated virus (AAV) is a non-lipid-enveloped virus often used to deliver DNA plasmids in vivo for gene therapy. Affinity chromatography is sometimes employed in its purification.

WO 2020/127505 discloses a method for removing nucleosomal contaminants from bioprocessing solutions. It is based on the inactivation or reduction of agents that inhibit the activity of nucleases, followed by treatment with enzymes to hydrolyze RNA and DNA. It describes using negatively charged polymers to extract peptides and proteins, as DNA- and RNA-binding proteins have a highly positive charge.

US 2018/0105554 discloses the use of dextran sulfate to enhance protein A affinity chromatography.

WO 2014/133459 discloses chromatographic purification of antibodies from chromatin-deficient cell culture harvest. Soluble or insoluble multivalent organic ions are contacted with an impure protein preparation prior to an adsorptive chromatography step.

### SUMMARY OF THE INVENTION

The invention pertains to a method in which an affinity chromatography column for purification of AAV is washed after sample loading with at least one step employing a charged compound containing 3 or more charges and at least one wash step employing a high concentration of a salt, where neither the charged compound or the salt are present in a concentration sufficient to elute the AAV.

The method for removal of contaminants in a sample of AAV provides the steps of:
loading the sample to an affinity chromatography column,
washing the column after the sample is loaded with a first buffer comprising a compound having more than three positive charges or more than three negative,
employing a second buffer having a salt concentration corresponding to a concentration of at least 0.25 M NaCl for displacing the compound
   wherein neither the compound nor the salt is present in a concentration sufficient to elute the AAV.

This approach surprisingly eliminates chromatin more effectively than washing with non-polymeric agents. By removing chromatin remnants so that they cannot interfere with later purification steps, it produces the further benefit of improving purification performance at those later steps, such as steps for removing empty capsids from mixed preparations of empty and full capsids.

“A compound having at least positive charges” can be an at least three times positively charged compound or a compound which becomes at least three times positively charged under conditions employed during washing the column, in particular a polymeric compound. “A compound having more than three positive charges” can be an at least four times positively charged compound or a compound which becomes at least four times positively charged under conditions employed during washing the column, in particular a polymeric compound. For example, the polymeric compound may be selected from the group consisting of pDADMAC (poly(diallyldimethylammonium chloride)), quaternary amino dextran, DEAE-dextran (diethylaminoethyl dextran), DEAE-cellulose, chitosan, polyethylenimine (PEI), and combinations thereof.

In another embodiment of the method of the invention the at least three times positively charged compound or the compound which becomes at least three times positively charged under conditions employed during washing the column can be a non-polymeric compound which particularly can be selected from the group consisting of bisbiguanide derivatives, such as 1,1′-(1,6-hexanediyl)bis{2-[N′-(2-ethylhexyl)carbamimidoyl]guanidine} (alexidine), 1′-hexamethylenbis[5-(4-chlorphenyl)biguanide] (chlorhexidine), polymyxin B, and combinations thereof.

“A compound having at least negative charges” can be an at least three times negatively charged compound or a compound which becomes at least three times negatively charged under conditions employed during washing the column. “A compound having more than three negative charges” can be an at least four times negatively charged compound or a compound which becomes at least four times negatively charged under conditions employed during washing the column. The negatively charged group may be provided by sulfo, phospho, carboxy groups, or mixtures thereof. The negatively charged compound may also be selected from the group consisting of polyvinyl sulfate, polyanetholesulfonic acid, dextran sulfate, polystyrene sulfuric acid, alginic acid, polyvinyl sulfate, pyrophosphate and combinations thereof.

In still another embodiment of the method of the invention the second buffer can comprise a salt selected from the group consisting of sodium chloride, potassium chloride, sodium acetate, potassium acetate, ammonium chloride, ammonium acetate, and combinations thereof. In particular, the salt of the second buffer can be present at a concentration of 0.25 M to 5.0 M, or 0.5 M to 4.0 M, or 1 M to 2 M, or 2 M to 3 M.

In a further embodiment of the method of the invention the first buffer may comprise a positively charged compound or negatively charged compound to elevate their conductivity into a range of about 1 mS/cm to about 25 mS/cm, 2 mS/cm to 20 mS/cm, 3 mS/cm to 15 mS/cm, 3 mS/cm to 10 mS/cm, or 4 mS/cm to 5 mS/cm.

In a still further embodiment of the method of the invention the first and/or the second buffer may comprise a chelating agent. The chelating agent may be particularly ethylenediaminetetraacetic acid (EDTA), or ethylene glycol-bis(O-aminoethyl ether)-N,N,N′,N′-tetraacetic acid (EGTA).

In a still further embodiment of the method of the invention the compound having three or more charges may be present at a concentration of 0.01% by weight to 5% by weight, 0.05% by weight to 1% by weight, or 0.1% by weight to 0.5% by weight.

In another embodiment of the method of the invention the sample of AAV can be a cell culture harvest, in particular a mammalian cell culture, an insect cell culture, or a cell lysate of such cell cultures. The AAV can be a serotype particularly selected from the group consisting of AAV1, AAV2, AAV3, AAV4, AAV5, AAV6, AAV7, AAV8, AAV9, AAV10, AAV11, a synthetic recombinant serotype, a recombinant hybrid serotype such as AAV2/8, AAV2/9, and combinations thereof.

In still another embodiment of the method of the invention the first and/or second buffer may comprise a nonionic or zwitterionic surfactant or detergent, in particular at a concentration in the range of 0.01 wt % to 1 wt %.

In yet another embodiment of the method of the invention the first and/or second buffer can comprise a nonionic chaotrope such as urea, in particular at a concentration in the range of 1 M to 10 M.

In a further embodiment of the method of the invention the first and/or second buffer may comprise an organic solvent such as methanol, ethanol, or isopropanol, in particular at a concentration in the range of 0.1 vol % to 20 vol %.

In preferred embodiments of the invention, no enzyme treatment with RNAase or DNAase or both is conducted on the sample prior to the step of loading the sample to the affinity chromatography column, preferably, no nuclease treatment and more preferably no enzyme treatment at all is done.

In preferred embodiments of the invention, no enzyme treatment with RNAase or DNAase or both is conducted on the sample while the sample is bound to the affinity chromatography column, preferably, no nuclease treatment and more preferably no enzyme treatment at all is done. Nuclease treatment in this context is the use of enzymes that individually or collectively enable simultaneous or successive hydrolysis of RNA and/or DNA, as used in WO 2020/127505.

### BRIEF DESCRIPTION OF THE FIGURES

FIG. 1 depicts a chromatogram of AAV purification by affinity chromatography not including the method of the invention.

FIG. 2 depicts analysis of the AAV affinity chromatography fractions showed in FIG. 1 by polyacrylamide gel electrophoresis.

FIG. 3 depicts residual DNA contamination remaining after purification by affinity chromatography as shown in FIG. 1.

FIG. 4 compares residual DNA contamination after conventional affinity chromatography with residual DNA contamination after affinity chromatography employing the method of the invention in example 9.

### DETAILED DESCRIPTION OF THE INVENTION

In one non-limiting example to illustrate the character of the method, an affinity chromatography column specific for AAV is loaded with an AAV containing sample. It may be washed briefly with equilibration buffer, if desired. The column is then washed with a positively charged polymer such as polyethyleneimine (PEI). The positively charged polymer is intended particularly to form strong complexes with contaminating nucleic acids such as DNA and RNA, thereby providing the nucleic acids with a surrogate for their initial binding to DNA-binding proteins. The binding of the positively charged polymer to DNA and its concomitant charge neutralization of DNA is believed to be a major contributor to the superior ability of the method over salt washes. After the charged polymer wash, the column is washed with a second wash containing a high concentration of a dissolved salt such as 1 M sodium chloride. The purpose of the second wash is to remove the charged polymer from the column so that the charged polymer will be absent from the eluted AAV.

In another non-limiting example to illustrate the character of the method, an affinity chromatography column specific for AAV is loaded with an AAV containing sample. It may be washed briefly with equilibration buffer, if desired. The column is then washed with a negatively charged polymer such as polyvinyl sulfate. The sulfo groups are intended particularly to form strong complexes with DNA-binding proteins such as histones and transcription factors, among others, thereby providing them with a surrogate for their initial binding to DNA. Although not bound by any theory it is believed that the binding of a negatively charge polymer to DNA-binding contaminants and its concomitant charge neutralization of DNA-binding proteins is a major contributor to the superior ability of the method over salt washes. After the charged polymer wash, the column is washed with a second wash containing a high concentration of a dissolved salt such as 1 M sodium chloride. The purpose of the second wash is to remove the charged polymer from the column so that the charged polymer will be absent from the eluted AAV.

In another non-limiting example to illustrate the character of the method, an affinity chromatography column specific for AAV is loaded with an AAV containing sample. It may be washed briefly with equilibration buffer, if desired. The column may then be washed with a solution of 1 M sodium chloride to destabilize chromatin, followed by a wash with a positively charged polymer, then a wash with salt to remove the positively charged polymer, then a wash with a negatively charged polymer, then a wash with salt to remove the negatively charged polymer.

In one embodiment, the charged compound contains three or more charged groups, either at least three positive or at least three negative charges. In one such embodiment, each of the charged groups is of a common basic structure, forming a polymer. In another such embodiment, the charged groups are part of a nonpolymeric structure.

In one embodiment, the charged compound is a priori at least three times positively charged or becomes at least three times positively charged under conditions employed during washing the column. In one such embodiment, the positive charge is endowed principally by the presence of quaternary amino groups. In one such embodiment, the charged polymer is pDADMAC (poly(diallyldimethylammonium chloride)) or quaternary amino dextran. In another embodiment, the positive charge is endowed principally by the presence of tertiary amino groups.

In one such embodiment the charged polymer is DEAE-dextran (diethylaminoethyl dextran) or DEAE-cellulose which becomes positively charged when in contact with water under conditions employed during washing the column, for example when simply dissolving in an aqueous buffer solution. In another embodiment, the positive charge is endowed principally by the presence of primary amino groups. In one such embodiment the charged polymer is chitosan. In another embodiment, the positive charge is principally by amino groups of mixed character including one or more of primary amino groups, secondary amino groups, tertiary amino groups and/or quaternary amino groups. In one such embodiment the charged polymer is PEI. In another embodiment, a non-polymeric positively charged compound is chlorhexidine. In a closely related embodiment, a non-polymeric positively charged compound is alexidine. In another closely related embodiment, a non-polymeric positively charged compound is TREN (tris(2-aminoethyl)amine) which becomes positively charged under conditions employed during washing the column. In another embodiment, a non-polymeric positively charged compound is polymyxin B, or another positively charged compound bearing at least three positively charged residues.

In one embodiment, the charged compound is a priori at least three times negatively charged or the compound becomes at least three times negatively charged under conditions employed during washing the column. In one such embodiment, the negative charge is endowed principally by the presence of sulfo groups. In one such embodiment, the charged polymer is polyvinyl sulfate. In another such embodiment, the charged polymer is polyanetholesulfonic acid. In another such embodiment, the charged polymer is dextran sulfate. In one such embodiment, the charged polymer is polystyrene sulfuric acid. In another embodiment, the negative charge is endowed principally by the presence of carboxyl groups. In one such embodiment, the negatively charge polymer is alginic acid. In another such embodiment, the negatively charged polymer is polyvinyl chloride. In a closely related embodiment, each negatively charged polymer subunit includes more than one carboxylic acid, such as two or three carboxylic acids residues. In one such embodiment, the negative charge is endowed principally by the presence of phospho groups. In another embodiment, the negatively charge polymer is of mixed character containing one or more sulfo, phospho, carboxyl, dicarboxyl, tricarboxyl, and or other negatively charged residues. In another embodiment, a nonpolymeric negatively charged compound is a pyrophosphate.

In one embodiment, a polymer backbone contains aliphatic sequences and/or aromatic residues to increase its hydrophobicity. In another embodiment, a polymer backbone lacks hydrophobic residues. In another embodiment, a nonpolymeric compound contains aliphatic sequences and/or aromatic residues.

In one embodiment, the salt employed to displace a charged polymer is sodium chloride, or potassium chloride, or sodium acetate, or potassium acetate, or ammonium chloride, or ammonium acetate, or another salt.

In one embodiment, the salt employed to displace a charged polymer is present at a concentration of 0.25 M to 5.0 M, or 0.5 M to 4.0 M, or 1 M to 2 M, or 2 M to 3 M.

In one embodiment, salt may be included in a wash solution containing positively charged compounds and/or negatively charged compounds to elevate their conductivity into a range of about 1 mS/cm to about 25 mS/cm, or 2 mS/cm to 20 mS/cm, or 3 mS/cm to 15 mS/cm, or 3 mS/cm to 10 mS/cm, or 4 mS/cm to 5 mS/cm. As a general matter, lower conductivity values will favor stronger interaction of positively charged compounds with nucleic acids, and stronger interaction of negatively charged compounds with DNA-binding proteins. High conductivity values will interfere with the ability of positively or negatively charged compounds to interact strongly with nucleic acids and/or DNA-binding proteins. Intermediate salt concentrations may provide better specificity since a moderate amount of salt will tend to block weak non-specific electrostatic interactions. Physiological conductivity will be a reasonable place to start since the sample and equilibration buffers are likely to be generally representative of physiological conditions. Physiological conductivity in the present context is considered to fall within the range of about 5 mS/cm to about 20 mS/cm or more narrowly in the range of about 10 mS/cm to about 15 mS/cm. Salt concentration can be adjusted up or down from that starting point.

In one embodiment, a washing solution contains a chelating agent to suppress non-specific interactions mediated through metal ions. In one such embodiment, a chelating agent is ethylenediaminetetraacetic acid (EDTA) at a concentration in the range from 0.1 mM to 100 mM, or 1 mM to 10 mM, or 2 mM to 5 mM. In a closely related embodiment, a chelating agent is ethylene glycol-bis(β-aminoethyl ether)-N,N,N′,N′,-tetraacetic acid (EGTA) at a concentration in the range from 0.1 mM to 100 mM, or 1 mM to 10 mM, or 2 mM to 5 mM. In another embodiment, another chelating agent is present at a concentration in the range from 0.1 mM to 100 mM, or 1 mM to 10 mM, or 2 mM to 5 mM.

In one embodiment, a charged compound with 3 or more charges is present at a concentration of 0.01% by weight to 5% by weight, or 0.05% by weight to 1% by weight, or 0.1% by weight to 0.5% by weight.

In one embodiment, washing with a charged polymer may be preceded by a salt wash step. In another embodiment, washing with a charged polymer may be followed with a salt wash step. In another embodiment, washing with a charged polymer may be preceded and followed by a salt-wash step.

In one embodiment, an affinity column may be washed with a single charged polymer. In one such embodiment, the polymer may be positively charged. In another such embodiment, the polymer may be negatively charged.

In one embodiment, an affinity column may be washed in sequence with a positively charged polymer and a negatively charged polymer. In one such embodiment, the positively charged polymer wash may occur first and the negatively charged polymer wash second. In another such embodiment, the negatively charged polymer wash may occur first and the positively charged polymer wash second. In another embodiment, the two polymer washes may be separated by a salt wash. It will be apparent to persons of knowledge in the art that the sequential combination of washing with a positively charged compound and a negative charged compound is expected to produce better results since it targets both nucleic acids and nucleic acid binding proteins. Nucleic acids do not exist separately from each other in process solutions but various subsets of chromatin tend to be more histone-rich or more DNA-rich on their surfaces. The positively charged compounds are expected to preferentially target nucleic acid-rich chromatin bodies. The negatively charged compounds are expected to preferentially target DNA-binding protein-rich chromatin bodies. It will be recognized that the hypothetical combination of positively charged polymers with negatively charged polymers is likely to be un-useful due to co-precipitation of the oppositely charged polymers with each other.

In one embodiment, the pH of any given wash step may range from a pH value in the range of about pH 4.5 to about pH 8.5 so long as the pH does not cause premature elution of the AAV.

In one embodiment, a washing solution may include a nonionic or zwitterionic surfactant or detergent at a concentration in the range of 0.01% by weight to 1% by weight. In a related embodiment, a wash solution may include a nonionic chaotrope such as a urea at a concentration in the range of 1 M to 10 M. In another related embodiment, a washing solution may include an organic solvent such as methanol, ethanol, or isopropanol at a concentration in the range of 0.1% by volume to 20% by volume. It will be recognized that some such additives will aid in inactivation of lipid enveloped viruses.

Persons of knowledge in the art will recognize the potential of the method of the invention to reduce virus and endotoxin contamination. Endotoxins and viruses are both known to become highly associated with chromatin so a method that particularly targets chromatin removal will be understood to also remove species that are associated with them.

In one embodiment, the method of the invention is used to improve affinity purification of AAV from a cell culture harvest. In one such embodiment, the cell culture is a mammalian cell culture. In a another such embodiment, the cell culture is an insect cell culture. In another embodiment, the method of the invention is used to improve affinity purification of AAV from a cell lysate. In one such embodiment, the cell culture is a mammalian cell culture. In a another such embodiment, the cell culture is an insect cell culture.

In one embodiment, the AAV serotype processed by the method of the invention may be AAV1, or AAV2, or AAV3, or AAV4, or AAV5, or AAV6, or AAV7, or AAV8, or AAV9, or AAV10, or AAV11, or another serotype. In another embodiment, the AAV serotype processed by the method of the invention may be a recombinant hybrid serotype like AAV2/8, or AAV2/9, or another hybrid serotype. In another embodiment, the AAV serotype processed by the method of the invention may be a synthetic recombinant serotype.

It will be recognized by persons of knowledge in the art that although AAV capsids of different serotypes share many basic physical and chemical similarities, they also exhibit significant variability with respect to their surface chemistry and purification characteristics. Accordingly, although the method of the invention will improve affinity purification of all serotypes, the specific conditions to obtain the best purification for any given serotype may differ. Surprisingly, the method of the invention is able to increase the amount of AAV recovered from the column, see example 9. One hypothesis for the unexpected differential was that the method of the invention liberated AAV particles that were too strongly bound to be removed from the experimental control. In the absence of a wash with buffer comprising a compound having at least three positive charges or three negative charges, these strongly bound AAV seem to stick to the column until it is cleaned under harsh conditions like a NaOH wash.

The invention is further explained by the following non-limiting examples.

### EXAMPLES

### Example 1 (Comparative)

An experimental control performed without the method of the invention

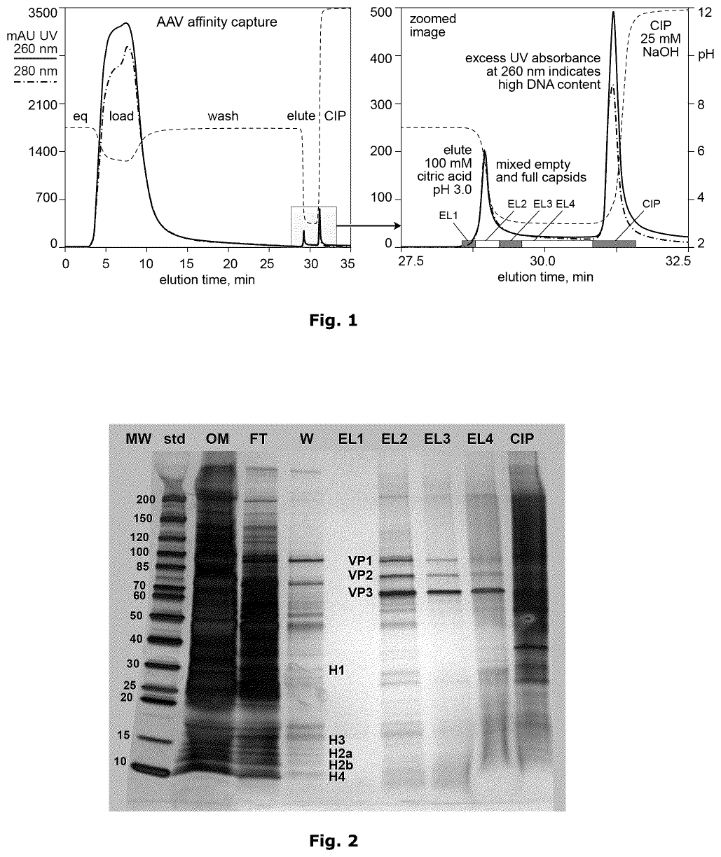

An affinity column specific for AAV was loaded with filtered lysate containing AAV8. The column was equilibrated with 50 mM sodium phosphate, 100 mM sodium chloride, pH 7.0. The sample was loaded. The column was washed with 50 mM sodium phosphate, 100 mM sodium chloride, pH 7.0, then eluted with a step to 100 mM acetic acid, pH 3.5. The column was cleaned with 20 mM sodium hydroxide. The elution profile is illustrated in FIG. 1. Analysis by polyacrylamide gel electrophoresis is illustrated in FIG. 2. Persistent contamination by chromatin is apparent from the presence of histone proteins. FIG. 3 illustrates analytical size exclusion chromatography (SEC) in which the sample was pre-treated with Picogreen Dye to increase sensitivity of DNA detection. Persistent chromatin contamination is apparent from the presence of DNA, particularly including high molecular weight (HMW) chromatin heteroaggregates.

### Example 2

The method of the invention using a positively charged polymer

A column for affinity chromatography capture of AAV is equilibrated with 50 mM sodium phosphate, 1 M sodium chloride, pH 7.0. A filtered cell culture harvest or lysate containing AAV is applied to the column, after which the column is washed for 5 CV with equilibration buffer. The column is washed for 10 CV with a solution containing 0.5% by weight PEI at a conductivity of 5 mS/cm and a pH of 7.0. The column is then washed again with equilibration buffer to clear PEI from the column. The AAV is then eluted with a step to 50 mM alanine, pH 2.3.

### Example 3

The method of example 2 is repeated except that the equilibration buffer contains 2 M sodium chloride.

### Example 4

The method of example 2 is repeated except that pDADMAC is substituted for PEI.

### Example 5

The method of example 4 is repeated except that a wash step with 5CV of 50 mM acetate pH 4.5 is conducted before elution.

### Example 6

The method of example 2 is repeated except that the equilibration buffer contains 100 mM sodium chloride, making it a physiological buffer. In this example, an additional wash solution if prepared containing 50 mM sodium phosphate, 1 M sodium chloride, pH 7.0. After loading the sample, a first 10 CV wash is applied with the 1 M sodium chloride buffer to destabilize chromatin associations with chromatin itself and with other surfaces it is associated with. The column is then washed with a second wash with 10 CV of 0.05% chlorhexidine digluconate in 50 mM sodium phosphate pH 7.0. The column is then washed with a third wash with the 1 M sodium chloride buffer, then finally washed with the original physiological buffer prior to elution.

### Example 7

The method of example 2 is repeated except substituting polyvinyl sulfate for PEI.

### Example 8

The method of the invention using a positively charged polymer and a negatively charged polymer. The method of example 6 is repeated except inserting a 10 CV wash with 0.05% by weight polyvinyl sulfate in 50 mM sodium phosphate, pH 7.0 after the second 1 M sodium chloride buffer wash. After the polyvinyl sulfate wash, the column is again washed with the 1 M sodium chloride buffer to clear the polyvinyl sulfate.

### Example 9

Paired comparison of affinity chromatography performance with and without the method of the invention. An experimental control was run following the same basic organization as FIG. 1. A new POROS GoPure AAVX (1 mL, P/N A36652, Lot 2004029) was equilibrated with 50 mM sodium phosphate, 100 mM sodium chloride, 1% sucrose, 0.1% poloxamer 188, pH 7.0. Clarified harvest containing about 1E+12 vg/mL was diluted 1:1 with column equilibration buffer. 30 mL of diluted harvest was loaded on the column at a flow rate of 0.33 CV/min. The column was then washed with 20 CV equilibration buffer to clear unbound sample components. The column was eluted with 0.1 M acetic acid, 1% sucrose, 0.1% poloxamer 188, pH 3.5. The elution fraction was neutralized with 1 M Tris, pH 9.0 (10% v/v). The column was cleaned with 20 mM NaOH.

A second new column was equilibrated and loaded exactly as the first. It was then washed with 10 CV 50 mM sodium phosphate, 0.05% polyethyleneimine (PEI), 0.1 M sodium chloride, 1% sucrose, 0.1% poloxamer 188, pH 7.0. It was washed again with 5 CV 50 mM sodium phosphate, 1 M sodium chloride, 1% sucrose, 0.1% poloxamer 188, pH 7.0 to displace remaining PEI from the previous washing step. It was washed again with 5 CV equilibration buffer to remove the excess salt and place the column in the same pre-elution conditions as the experimental control. This was done to facilitate objective comparison of the size of the elution peaks between the two experiments. The column was then eluted, the elution peak was neutralized, and the column was cleaned as was the experimental control.

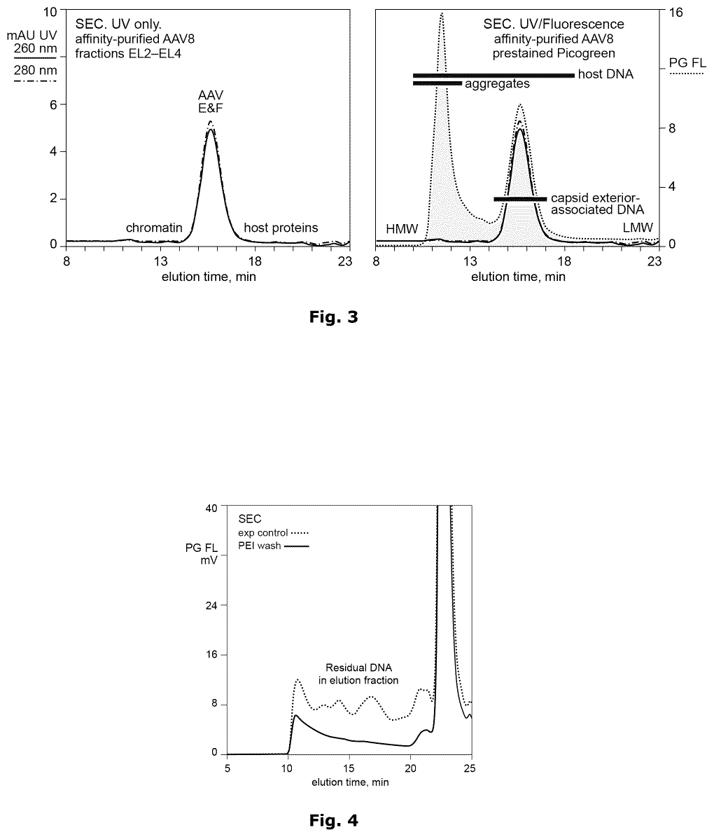

The most striking result from employing the method of the invention was that the size of the AAV2 elution peak area was roughly doubled over the experimental control. This was confirmed by PCR testing of the two elution peaks. The peak from the experimental control contained 2.38 E+11 vg/mL. The elution peak from the experiment using the method of the invention contained 4.18 E+11 vg/mL. This result was notable since identical new columns had been loaded with identical volumes of the identical sample, creating an expectation that the elution peaks should have contained the same amount of AAV. One hypothesis for the unexpected differential was that the method of the invention liberated AAV particles that were too strongly bound to be removed from the experimental control in the absence of the PEI-salt wash. For this to be true would create an expectation that the size of the column-cleaning peak would be reduced in the test experiment, as was observed. These results suggest that the method of the invention works by destabilizing the association of AAV2 with DNA, changing the conformation of the DNA aggregates that are associated with the AAV. Further support for this rationale is seen in FIG. 4, which illustrates the size distribution of the DNA in the AAV2 eluted from both columns. In both cases, a sample of the respective eluate was pre-stained with Picogreen, which produces green fluorescence upon intercalation into DNA. Both profiles showed contaminating DNA in the elution fractions, spanning a broad range of sizes. However, the amount of DNA in the eluted AAV from the experiment employing the method of the invention was less than half the amount observed in the AAV eluted after using a traditional washing strategy. It may be noteworthy that the result of increasing chromatographic recovery by manipulation of chromatin has been observed when removing chromatin in advance of chromatography, rather than during chromatography [2-4]. However, the degree of improvement is much more modest than observed in the present experiment: recovery increased 5%-10% versus the 100% increase achieved using the method of the invention. It will be appreciated by persons of skill in the art that the ability of the method of the invention to double product recovery has important ramifications for industrial purification of AAV.

### Example 10

The method of example 9 is repeated except substituting 2 M sodium chloride for 1 M sodium chloride in the post-polymer wash step.

### Example 11

The method of example 9 is repeated except substituting 50 mM alanine pH 2.3 for 50 mM acetic acid, pH 3.5

### Example 12

The method of example 9 is repeated except substituting 0.5% PEI for 0.05% PEI.

### REFERENCES

All references cited anywhere in the specification are incorporated by reference to the full extent to which the incorporation is not inconsistent with the express teachings herein.
[1] P Gagnon, Purification Tools for Monoclonal Antibodies, 1996. Validated Biosystems, Tucson.
[2] A Shukla, P Hinckley, Host cell protein clearance during protein A chromatography: development of an improved column wash step, Biotechnol Prog 24 (2008) 1115-1121.
[3] P Gagnon, P Toh, J Lee, High productivity purification of immunoglobulin G monoclonal antibodies on starch-coated magnetic nanoparticles by steric exclusion of polyethylene glycol, J Chromatogr A 1324 (2014) 171-180.
[4] P Gagnon, R Nian, J Lee, L Tan, SM Abdul Latiff, CL Lim, C Chuah, X Bi, YS Yang, W Zhang, HT Gan, Nonspecific interactions of chromatin with immunoglobulin G and protein A, and their impact on purification performance, J Chromatogr A 1340 (2014) 68-78.
[5] R Nian, W Zhang, L Tan, J Lee, X Bi, YS Yang, HT Gan, P Gagnon, Advance chromatin extraction improves capture performance of protein A affinity chromatography, J Chromatogr A 1431 (2016) 1-7.
[6] M McPhillips, K Ozato, A McBride, Interaction of bovine papillomavirus E2 protein with Brd4 stabilizes its association with chromatin, J Virol 79 (2005) 8920-8932.
[7] D Knipe, P Lieberman, J Jung, A McBride, K Morris, M Ott, D Margolis, A Nieto, M Nevels, R Parks, T Kristie, Snapshots: Chromatin control of viral infection, Virology 435 (2013) 141-156.
[8] P Lieberman, Chromatin regulation of virus infection, Trends Microbiol 14 (2006) 132-140.
[9] L Augusto, P Decottiginies, M Synguelakis, M Nicaise, P Lee Marechal, R Chaby, Histones: a novel class of liposaccharide-binding molecules, Biochemistry 42 (2003) 3929-3938.

1. A method for removal of contaminants in a sample of AAV comprising the steps of
loading the sample to an affinity chromatography column;
washing the column after the sample is loaded with a first buffer comprising a compound having at least three positive charges or at least three negative charges; and
employing a second buffer having a salt concentration corresponding to a concentration of at least 0.25 M NaCl for displacing the compound,
wherein neither the compound nor the salt is present in a concentration sufficient to elute the AAV.

2. The method of claim 1 wherein the compound having at least three positive charges is a positively charged compound or a compound which becomes positively charged under conditions employed during washing.

3. The method of claim 2 wherein the positively charged compound or the compound which becomes positively charged under conditions employed during washing the column is a polymeric compound.

4. The method of claim 2 wherein the positively charged compound or the compound which becomes positively charged under conditions employed during washing the column is a non-polymeric compound.

5. The method of claim 1 wherein the compound having at least three negative charges is a negatively charged compound or the compound becomes negatively charged under conditions employed during washing the column.

6. The method of claim 5 wherein the negative charge of the negatively charged compound is provided by sulfo groups, phosphor groups, carboxy groups, or mixtures thereof.

7. The method of claim 5 wherein the negatively charged compound is selected from the group consisting of polyvinyl sulfate, polyanetholesulfonic acid, dextran sulfate, polystyrene sulfuric acid, alginic acid, polyvinyl sulfate, pyrophosphate and combinations thereof.

8. The method of claim 1 wherein the second buffer comprises a salt selected from the group consisting of sodium chloride, potassium chloride, sodium acetate, potassium acetate, ammonium chloride, ammonium acetate, and combinations thereof.

9. The method of claim 1 wherein the first buffer comprises a positively charged compound or negatively charged compound to elevate their conductivity into a range of about 1 mS/cm to about 25 mS/cm.

10. The method of claim 1 wherein the first and/or the second buffer comprise a chelating agent selected from ethylenediaminetetraacetic acid (EDTA), and ethylene glycol-bis(p-aminoethyl ether)-N,N,N′,N′-tetraacetic acid (EGTA).

11. The method of claim 1 wherein the compound having three or more charges is present at a concentration of 0.01% by weight to 5% by weight.

12. The method of claim 1 wherein the sample of AAV is a cell culture harvest.

13. The method of claim 1 wherein the AAV is a serotype selected from the group consisting of AAV1, AAV2, AAV3, AAV4, AAV5, AAV6, AAV7, AAV8, AAV9, AAV10, AAV11, a synthetic recombinant serotype, a recombinant hybrid serotype, and combinations thereof.

14. The method of claim 1 wherein
i) the first and/or second buffer comprises a nonionic or zwitterionic surfactant or detergent, and/or
ii) wherein the first and/or second buffer comprises a nonionic chaotrope such as urea.

15. The method of claim 1 wherein the first and/or second buffer comprises an organic solvent.

16. The method of claim 1 wherein no enzyme treatment with RNAase or DNAase or both is conducted on the sample prior to the step of loading the sample to the affinity chromatography column.

17. The method of claim 1 wherein no enzyme treatment with RNAase or DNAase or both is conducted on the sample while the sample is bound to the affinity chromatography column.

18. The method of claim 16 wherein no nuclease treatment or no enzyme treatment is done.

19. The method of claim 17 wherein no nuclease treatment or no enzyme treatment is done.

20. The method of claim 3 wherein the polymeric compound is selected from the group consisting of poly(diallyldimethylammonium chloride), quaternary amino dextran, diethylaminoethyl dextran, diethylaminoethyl cellulose, chitosan, polyethylenimine, and combinations thereof.

21. The method of claim 4 wherein the non-polymeric compound, is selected from the group consisting of bisbiguanide derivatives, such as 1,1′-(1,6-hexanediyl)bis{2-[N′-(2-ethylhexyl)carbamimidoyl]guanidine} (alexidine), 1′-hexamethylenbis[5-(4-chlorphenyl)biguanide] (chlorhexidine), polymyxin B, and combinations thereof.

22. The method of claim 12 wherein the culture harvest is a mammalian cell culture, an insect cell culture, or a cell lysate of such cell cultures.
